(19) United States
(12) Patent Application Publication
(10) Pub. No.: US 2021/0393684 A1
(43) Pub. Date: Dec. 23, 2021

(54) Adoptive T-Cell Therapy for CMV Infection and CMV-Associated Diseases

(71) Applicant: The Council of the Queensland Institute of Medical Research (QIMR), Hertson, Queensland (AU)

(72) Inventors: Rajiv Khanna, Herston (AU); Corey Smith, Ashgrove (AU)

(73) Assignee: The Council of the Queensland Institute of Medical Research (QIMR), Hertson, Queensland (AU)

(21) Appl. No.: 17/056,103

(22) PCT Filed: May 16, 2019
(86) PCT No.: PCT/IB2019/000588

§ 371 Date: Jul. 1, 2021

Related U.S. Application Data
(60) Provisional application No. 62/673,260, filed on May 18, 2018.

(51) Int. Cl. A61K 35/17; C07K 14/005; C12N 5/0783; A61P 31/20; A61K 39/245; C07K 14/74

(52) U.S. Cl. CPC A61K 35/17; C07K 14/005; C12N 5/0636; A61P 31/20; A61K 39/245; A61K 2039/5158; C12N 2501/998; C12N 2501/2302; C12N 2710/16122; C12N 2710/16134; C07K 14/70539

(57) ABSTRACT

Provided herein are immunogenic polypeptides, compositions, and methods related to the development of CMV-specific prophylactic and/or therapeutic immunotherapy based on T cell epitopes (e.g., CMV epitopes) that are recognized by cytotoxic T cells (CTLs) and can be employed in the prevention and/or treatment of CMV infection, reactivation, and/or disease (e.g., CMV-associated end organ disease), especially in solid organ transplant recipients.

### RELATED APPLICATIONS

This application is a national phase entry pursuant to 35 U.S.C. § 371 of International Application No. PCT/IB2019/000588, filed 16 May 2019, which claims the benefit of priority to U.S. Provisional Patent Application Ser. No. 62/673,260 filed May 18, 2018, both of which are incorporated by reference in their entirety.

### BACKGROUND

Herpesviruses represent a large and near ubiquitous family of eukaryotic viruses associated with a variety of animal and human diseases. Herpesviridae share several common structures, e.g., double-stranded, linear DNA genomes, and a virion comprising an icosahedral capsid, which is itself wrapped in a layer of viral tegument and a lipid bilayer (the viral envelope). In addition, herpesviruses comprise characteristic and highly conserved glycoproteins, carried on the lipid bilayer envelope of the herpesvirus virion. At least some of these glycoproteins play a role in the initial attachment of virus to the cell surface and subsequent penetration into cells.

Members of the herpesvirus family represent important human pathogens, among which is human cytomegalovirus (CMV). Cytomegalovirus can be found universally throughout all geographic locations and socioeconomic groups, infecting between 60% to 90% of individuals. In healthy individuals, after primary infection, CMV establishes a latent state with periodical reactivation and shedding from mucosal surfaces and may be accompanied with clinical symptoms of a mononucleosis-like illness, similar to that caused by Epstein-Barr virus, but is generally asymptomatic. CMV employs a multitude of immune-modulatory strategies to evade the host immune response. Examples of such strategies include inhibition of interferon (IFN) and IFN-stimulated genes, degradation of HLA to prevent antigen presentation to cytotoxic T-cells, and modulation of activating and inhibitory ligands to prevent natural killer (NK) cell function.

However, under certain conditions, CMV can cause significant morbidity and mortality. For example, the clinical management of CMV infection in solid organ transplant (SOT) recipients remains a major challenge. The incidence of early CMV-associated complications in SOT recipients has significantly reduced since the advent of virostatic therapy based on ganciclovir. The inhibition of viral reactivation by either the prophylactic or pre-emptive administration of ganciclovir has therefore become critical in the prevention of CMV-associated disease. However, late CMV reactivation can be more problematic to manage, especially in patients who are unable to reconstitute anti-viral T-cell immunity. Furthermore, the emergence of ganciclovir-resistant CMV reactivation or disease poses major difficulties in clinical management, with significant morbidity and mortality due to drug-associated toxicity, immunomodulatory impact and allograft loss.

Alternative safe and effective therapeutic options for ganciclovir-resistant CMV are lacking. Additional anti-viral management strategies, using foscarnet or cidofovir, are associated with nephrotoxicity, and require intravenous administration and hospitalisation. Genes conferring resistance to ganciclovir are also associated with resistance to foscarnet and cidofovir. Reduction in immunosuppression can be used to improve viral control, but increases the risk of graft rejection.

Thus, there is a great need for new and improved methods and compositions for the treatment of CMV infection, reactivation, and associated complications and diseases in SOT recipients and other patients with CMV-related disease.

### SUMMARY

Provided herein are immunogenic polypeptides, compositions, and methods related to the development of CMV-specific prophylactic and/or therapeutic immunotherapy based on T cell epitopes (e.g., CMV epitopes) that are recognized by cytotoxic T cells (CTLs) and can be employed in the prevention and/or treatment of CMV infection, reactivation, and/or disease (e.g., CMV-associated end organ disease), especially in solid organ transplant recipients. In some embodiments, the CMV infection, reactivation, and/or disease is persistent. In certain embodiments the CMV infection, reactivation, and/or disease is resistant to anti-viral therapy.

Also provided herein are pools of immunogenic peptides comprising HLA class I and class II-restricted Cytomegalovirus (CMV) peptide epitopes capable of inducing proliferation of peptide-specific T cells. In some embodiments, the pool of immunogenic peptides comprises at least one of the epitope amino acid sequences set forth in SEQ ID NOs. 25 to 29, or combinations thereof. In certain embodiments, the peptide pool comprises at least one peptide epitope derived from each of the CMV antigens pp50, pp65, IE-1, gB and gH. Preferably, such immunogenic peptide pools further comprise at least one of the CMV peptide epitope amino acid sequences set forth in Table 1. More preferably, the immunogenic peptide pools of the invention comprise each of the CMV peptide epitope amino acid sequences set forth in Table 1. In some embodiments, each of the epitopes of the immunogenic peptide pools disclosed herein are restricted by any one of the HLA specificities selected from HLA-A*01:01, -A*02:01, -A*23:01, -A*24:02, -B*07:02, -B*08:01, -B*18:01, -B*35:01, -B*35:08, -B*40:01, -B*40:02, -B*41.01, -B*44:02, -C*06:02, -C*07:02, -DRB1*01:01, -DRB1*03:01, -DRB1*04:01, -DRB1*07, or -DRB1*11:01

In some aspects, provided herein are methods of producing a preparation comprising polyfunctional, CMV-specific cytotoxic T cells (CTLs), comprising the steps of a) isolating a sample comprising CTLs; b) exposing said sample to the pool of immunogenic peptides of any one of claims 1 to 6; and c) harvesting the CTLs. In certain embodiments, the pool of immunogenic peptides consists essentially of each of the CMV peptide epitope amino acid sequences set forth in Table 1. In some embodiments, the sample comprising CTLs comprises peripheral blood mononuclear cells (PBMCs) from a healthy donor. In some such embodiments, the donor is immunocompromised. In certain embodiments, the donor is undergoing immunosuppressive therapy. Preferably, the donor is a solid organ transplant recipient. In certain preferred embodiments, the donor is receiving anti-viral therapy.

In some embodiments, the exposed sample of step b) is incubated for at least 14 days. Cytokines may be employed in the process of the instant invention and may include, without limitation, IL-1, IL-2, IL-4, IL-6 IL-7, IL-12, IL-15, and/or IL-21. For Example, the exposed sample of step b) may be incubated with IL-21 on day 0. In some such embodiments, the exposed sample of step b) is incubated with IL-2 on day 2. Preferably, the sample is incubated with IL-2 every three days.

In certain aspects of the invention, provided herein are methods of treating or preventing CMV infection in a subject, comprising administering to the subject the CTLs, or compositions thereof, produced by the methods disclosed herein. In some embodiments the subject is suffering from CMV reactivation or a CMV-associated condition (e.g., CMV-associated end organ disease), or at risk thereof. In certain preferred embodiments, the subject has received a solid organ transplant. Also provided herein are methods of reducing or eliminating the need for anti-viral therapy in a subject that has received a solid organ transplant, such methods comprising administering to the subject the CTLs produced by the methods disclosed herein.

### BRIEF DESCRIPTION OF THE DRAWINGS

FIG. 1 shows the phenotypic and functional characteristics of CMV-specific T-cells expanded for adoptive immunotherapy. (A) The phenotypic characteristics of CMV peptide pool-expanded T-cells were assessed using standard TBNK (T-cell, B-cell, NK-cell) analysis, measuring the surface expression of CD3 (T-cells), CD8 (CD8+ T-cells), CD4 (CD4+ T-cells), CD16 and CD56 (NK-cells) and CD19 (B-cells). (B) PBMC (ex vivo; prior to exposure to peptides) or expanded T-cells (Day 14) were assessed for the intracellular production of IFN-γ following re-stimulation with the CMV peptide pool or with individual HLA-matched peptides. The data represent the proportion of CD8+ T-cells producing IFN-γ. (C) Comparison of CMV-specific T-cell responses generated from either kidney or heart/lung transplant patients (D) Comparison of CMV-specific T-cell responses generated from either CMV-seronegative recipients (R-) or CMV-seropositive recipients (R+). (E) CMV peptide pool-stimulated T cells were assessed for intracellular cytokine production (IFN-γ, TNF, IL-2) and degranulation (CD107a) following recall with the CMV peptide pool. The data represent the proportion of the total antigen-specific T-cells producing each combination of effector functions (i.e., polyfunctionality).

FIG. 2 shows immunological and virological effects following adoptive cellular therapy. (A) PBMC samples from patients before and after T-cell therapy were assessed for IFN-γ-producing CMV-specific T-cells following stimulation with the CMV peptide pool. The data represent an overlay of the number of IFN-γ-producing CD8+ T-cells and the CMV load in copies/mL from four patients who showed a response to therapy. The shaded area indicates the time period prior to adoptive T-cell therapy and the arrows represent T-cell infusions. (B) Polyfunctionality, i.e., cytokine production (IFN-γ, TNF, IL-2) and degranulation (CD107a), was assessed on PBMC samples following stimulation with the CMV peptide pool. Heat-maps represent the proportion of total antigen-specific T cells producing each combination of effector functions.

FIG. 3 shows polychromatic profiling of T-cell phenotype. Representative t-distributed stochastic neighbor embedding (tSNE) analysis in the upper panels of FIG. 3 show the expression of T cell phenotype markers and CMV-specific T cells (VTE) pre-therapy and post-therapy in patient P1553PAH08, and demonstrate an increase in the expression of CD57. Data in the bottom panels of FIG. 3 represent an overlay of the proportion of CD8+ T-cells expressing CD57 post T cell therapy and the percentage CMV-specific IFN-γ producing cells in three SOT recipients (P1553PAH08, 1553PCH02 and 1553PCH04) who responded to adoptive T cell therapy and one SOT recipient (P1553RAH01) who failed to show any clinical response.

### DETAILED DESCRIPTION

### General

The reconstitution of CMV immunity through the administration of CMV-specific T-cells offers an attractive option to enhance the control of CMV. Using a plurality of epitopes from multiple CMV antigens as disclosed herein can induce a broad repertoire of virus-specific immune responses to provide more effective protection against virus-associated pathogenesis. Most preferably, the present disclosure relates to the stimulation and expansion of polyfunctional T-cells, i.e., those T cells that are capable of inducing multiple immune effector functions, that provide a more effective immune response to a pathogen than do cells that produce, for example, only a single immune effector (e.g. a single biomarker such as a cytokine or CD107a). Less-polyfunctional, monofunctional, or even “exhausted” T cells may dominate immune responses during chronic infections, thus negatively impacting protection against virus-associated complications.

However, in the case of SOT recipients, autologous immune cells from heavily immunosuppressed individuals are required to generate an effective T-cell therapy. While showing some promising results with autologous CMV-specific T-cell therapy in a SOT patient, a previous case study also raised potential safety concerns (Brestrich et al. (2009) Am J Transplant 9(7): 1679-84). As a consequence, the development of this approach has been limited due to the perceived difficulties in generating T-cells from highly immunosuppressed subjects (e.g., SOT recipients), and the potential risks associated with graft rejection following T-cell administration.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen-presenting cell provided herein and/or a CTL provided herein.

As used herein, the term “subject” or “recipient” means a human or non-human animal selected for treatment or therapy.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The term “binding” or “interacting” refers to an association, which may be a stable association, between two molecules, e.g., between a TCR and a peptide/MHC, due to, for example, electrostatic, hydrophobic, ionic and/or hydrogen-bond interactions under physiological conditions.

As used herein, “specific binding” refers to the ability of a TCR to bind to a peptide presented on an MHC (e.g., class I MHC or class II MHC). Typically, a TCR specifically binds to its peptide/MHC with an affinity of at least a K_{D} of about 10⁻⁴ M or less, and binds to the predetermined antigen/binding partner with an affinity (as expressed by K_{D}) that is at least 10 fold less, at least 100 fold less or at least 1000 fold less than its affinity for binding to a non-specific and unrelated peptide/MHC complex (e.g., one comprising a BSA peptide or a casein peptide).

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid; or cells from any time in gestation or development of the subject.

As used herein, the term “cytokine” refers to any secreted polypeptide that affects the functions of cells and is a molecule which modulates interactions between cells in the immune, inflammatory or hematopoietic response. A cytokine includes, but is not limited to, monokines and lymphokines, regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epidermal keratinocytes and B-lymphocytes. Lymphokines are generally referred to as being produced by lymphocyte cells. Examples of cytokines include, but are not limited to, Interleukin-1 (IL-1), Interleukin-2 (IL-2), Interleukin-6 (IL-6), Interleukin-8 (IL-8), Tumor Necrosis Factor-alpha (TNFα), and Tumor Necrosis Factor beta (TNFβ).

The term “epitope” means a protein determinant capable of specific binding to an antibody or TCR. Epitopes usually consist of chemically active surface groupings of molecules such as amino acids or sugar side chains. Certain epitopes can be defined by a particular sequence of amino acids to which an antibody is capable of binding.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Peptides

Provided herein are peptides comprising herpesvirus epitopes that are recognized by cytotoxic T lymphocytes (CTLs) and that are useful in the prevention and/or treatment of CMV infection, reactivation, and/or disease of CMV infection and/or cancer (e.g., end-organ disease in solid organ transplant recipients). In certain embodiments, the CMV epitope is an epitope listed in Table 1.

TABLE 1
Exemplary CMV epitopes

| | HLA | | | SEQ |
|---|---|---|---|---|
| Epitope | restric- | HCMV | | ID |
| Sequence | tion | antigen | Code | NO: |
| VTEHDTLLY | A*01:01 | pp50 | VTE | 1 |
| YSEHPTFTSQY | A*01:01 | pp65 | YSEH | 2 |
| NLVPMVATV | A*02:01 | pp65 | NLV | 3 |
| VLEETSVML | A*02:01 | IE-1 | VLE | 4 |
| YILEETSVML | A*02:01 | IE-1 | YIL | 5 |
| AYAQKIFKIL | A*23:01 | IE-1 | AYA | 6 |
| | A*24:02 | | | |
| QYDPVAALF | A*24:02 | pp65 | QYD | 7 |
| TPRVTGGGAM | B*07:02 | pp65 | TPR | 8 |
| RPHERNGFTVL | B*07:02 | pp65 | RPH | 9 |
| ELRRKMMYM | B*08:01 | IE-1 | ELR | 10 |
| ELKRKMIYM | B*08:01 | IE-1 | ELK | 11 |
| QIKVRVDMV | B*08:01 | IE-1 | QIK | 12 |
| DELRRKMMY | B*18:01, | IE-1 | DEL | 13 |
| | B*44:02 | | | |
| IPSINVHHY | B*35:01 | pp65 | IPS | 14 |
| CPSQEPMSIYVY | B*35:08 | pp65 | CPS | 15 |
| CEDVPSGKL | B*40:01 | pp65 | CED | 16 |
| HERNGFTVL | B*40:01, | pp65 | HER | 17 |
| | B*40:02 | | | |
| EEAIVAYTL | B*40:01, | IE-1 | EEA | 18 |
| | B*44:02 | | | |
| QEFFWDANDIY | B*44:02 | pp65 | QEF | 19 |
| TRATKMQVI | C*06:02 | pp65 | TRA | 20 |
| YAYIYTTYL | B*41:01 | gB | YAY | 21 |
| QAIRETVEL | B*35:01 | pp65 | QAI | 22 |
| CRVLCCYVL | C*07:02 | pp65 | CRV | 23 |
| HELLVLVKKAQL | DRB1*11:01 | gH | HELL | 24 |
| DYSNTHSTRYV | DRB1*07 | gB | DYSN | 25 |
| QEFFWDANDIYRIFA | DRB3*01:01 | pp65 | QEFF | 26 |
| CMLTITTARSKYPYH | DRB1*04:01 | gH | CMLT | 27 |
| PLKMLNIPSINVHHY | DRB1*01:01 | pp65 | PLKM | 28 |
| EHPTFTSQYRIQGKL | DRB1*11:01 | pp65 | EHPT | 29 |
| AGILARNLVPMVATV | DRB1*03:01 | pp65 | AGIL | 30 |
| KARAKKDELR* | HLA-B*31:01 | IE-1 | KAR | 31 |

*For patient P1553PAH01, the CMV peptide pool was supplemented with the IE-1-encoded HLA-B*31:01-restricted epitope KARAKKDELR (KAR).

In certain aspects, provided herein are pools of immunogenic peptides comprising HLA class I and class II-restricted Cytomegalovirus (CMV) peptide epitopes capable of inducing proliferation of peptide-specific T cells. In some embodiments, the pool of immunogenic peptides comprises at least one of the epitope amino acid sequences set forth in SEQ ID NOs. 25 to 29, or combinations thereof. In some such embodiments, the peptide pool comprises at least one peptide epitope derived from each of the CMV antigens pp50, pp65, IE-1, gB and gH. Preferably, the pool of immunogenic peptides further comprises at least one of the CMV peptide epitope amino acid sequences set forth in Table 1, or a combination thereof. Most preferably, such peptide pools comprise each of the CMV peptide epitope amino acid sequences set forth in Table 1.

By “HLA restriction (i.e., MHC restriction), it is meant that a given T cell will recognize and respond to the peptide, only when it is bound to a particular HLA molecule. In some embodiments, each of the epitopes of the immunogenic peptide pools disclosed herein are restricted by any one of the HLA specificities selected from HLA-A*01:01, -A*02:01, -A*23:01, -A*24:02, -B*07:02, -B*08:01, -B*18:01, -B*35:01, -B*35:08, -B*40:01, -B*40:02, -B*41.01, -B*44:02, -C*06:02, -C*07:02, -DRB1*01:01, -DRB1*03:01, -DRB1*04:01, -DRB1*07, or -DRB1*11:01.

Most preferably, the immunogenic peptides, and pools thereof, are capable of inducing proliferation of peptide-specific cytotoxic T cells (CTLs).

In some embodiments, the peptides provided herein are full length CMV polypeptides. In some embodiments, the peptides provided herein comprise less than 100, 90, 80, 70, 60, 50, 40, 30, 25, 20, 15 or 10 contiguous amino acids of the CMV viral polypeptide. In some embodiments, the peptides provided herein comprise two or more of the CMV epitopes listed in Table 1. For example, in some embodiments, the peptides provided herein comprise two or more of the CMV epitopes listed in table 1 connected by polypeptide linkers. In some embodiments, the peptide provided herein comprises at least 2, 3, 4, 5, 6, 7, 8, 9, 10, 11, 12, 13, 14, 15, 16, 17, 18, 19 or all of the epitopes listed in Table 1.

In some embodiments, the sequence of the peptides comprise a CMV viral polypeptide sequence except for 1 or more (e.g., 1, 2, 3, 4, 5, 6, 7, 8, 9, 10 or more) conservative sequence modifications. As used herein, the term “conservative sequence modifications” is intended to refer to amino acid modifications that do not significantly affect or alter the interaction between a T-cell receptor (TCR) and a peptide containing the amino acid sequence presented on a major histocompatibility complex (MI-IC). Such conservative modifications include amino acid substitutions, additions (e.g., additions of amino acids to the N or C terminus of the peptide) and deletions (e.g., deletions of amino acids from the N or C terminus of the peptide). Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the peptides described herein can be replaced with other amino acid residues from the same side chain family and the altered peptide can be tested for retention of TCR binding using methods known in the art. Modifications can be introduced into an antibody by standard techniques known in the art, such as site-directed mutagenesis and PCR-mediated mutagenesis.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

Also provided herein are chimeric or fusion proteins. As used herein, a “chimeric protein” or “fusion protein” comprises a peptide(s) provided herein (e.g., those comprising an epitope listed in Table 1) linked to a distinct peptide to which it is not linked in nature. For example, the distinct peptide can be fused to the N-terminus or C-terminus of the peptide either directly, through a peptide bond, or indirectly through a chemical linker. In some embodiments, the peptide of the provided herein is linked to polypeptides comprising other CMV epitopes. In some embodiments, the peptide provided herein is linked to peptides comprising epitopes from other viral and/or infectious diseases. In some embodiments, the peptide provided herein is linked to a peptide encoding a cancer-associated epitope.

A chimeric or fusion peptide provided herein can be produced by standard recombinant DNA techniques. For example, DNA fragments coding for the different peptide sequences can be ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Similarly, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, Ausubel et al., eds., John Wiley & Sons: 1992). Moreover, many expression vectors that already encode a fusion moiety are commercially available.

In some aspects, provided herein are cells that present a peptide described herein (e.g., a peptide comprising an epitope listed in Table 1). In some embodiments, the cell is a mammalian cell. The cell may be an antigen-presenting cell (APC) (e.g., an antigen presenting t-cell, a dendritic cell, a B cell, a macrophage or am artificial antigen presenting cell, such as aK562 cell). A cell presenting a peptide described herein can be produced by standard techniques known in the art. For example, a cell may be pulsed to encourage peptide uptake. In some embodiments, the cells are transfected with a nucleic acid encoding a peptide provided herein.

In some aspects, provided herein are methods of producing antigen-presenting cells (APCs), comprising pulsing a cell with the peptides described herein. Exemplary methods for producing antigen presenting cells can be found in WO2013088114, hereby incorporated in its entirety.

The peptides described herein can be isolated from cells or tissue sources by an appropriate purification scheme using standard protein purification techniques, can be produced by recombinant DNA techniques, and/or can be chemically synthesized using standard peptide synthesis techniques. The peptides described herein can be produced in prokaryotic or eukaryotic host cells by expression of nucleotides encoding a peptide(s) of the present invention. Alternatively, such peptides can be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N. Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Cells

In some aspects, provided herein are antigen-presenting cells (APCs) that express on their surface an MHC that present one or more peptides comprising a CMV epitope described herein (e.g., APCs that present one or more of the CMV epitopes listed in Table 1). In some embodiments, the MHC is a class I MHC. In some embodiments, the MHC is a class II MHC. In some embodiments, the class I MHC has an α chain polypeptide that is HLA-A, HLA-B, HLA-C, HLA-E, HLA-F, HLA-g, HLA-K or HLA-L. In some embodiments, the class II MHC has an a chain polypeptide that is HLA-DMA, HLA-DOA, HLA-DPA, HLA-DQA or HLA-DRA. In some embodiments, the class II MHC has a (3 chain polypeptide that is HLA-DMB, HLA-DOB, HLA-DPB, HLA-DQB or HLA-DRB.

In some embodiments, the APCs are B cells, antigen-presenting T-cells, dendritic cells, or artificial antigen-presenting cells (e.g., aK562 cells). Dendritic cells for use in the process may be prepared by taking PBMCs from a patient sample and adhering them to plastic. Generally, the monocyte population sticks and all other cells can be washed off. The adherent population is then differentiated with IL-4 and GM-CSF to produce monocyte derived dendritic cells. These cells may be matured by the addition of IL-1(3, IL-6, PGE-1 and TNF-α (which upregulates the important co-stimulatory molecules on the surface of the dendritic cell) and are then transduced with one or more of the peptides provided herein.

In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference.

In certain aspects, provided herein are methods of generating APCs that present the one or more of the CMV epitopes described herein comprising contacting an APC with a peptide comprising a CMV epitope, or a pool of CMV epitope peptides as described herein and/or with a nucleic acid encoding one or more CMV epitope peptides described herein. In some embodiments, the APCs are irradiated.

In certain aspects, provided herein are T-cells (e.g., CD4 T-cells and/or CD8 T-cells) that express a TCR (e.g., an αβ TCR or a γδ TCR) that recognizes a peptide described herein (a peptide comprising a CMV epitope listed in Table 1) presented on a MHC (e.g., HLA-restricted). In some embodiments, the T-cell is a CD8+ T-cell (a CTL) that expresses a TCR that recognizes a peptide described herein presented on a class I MHC (e.g., HLA-A, -B, and -C). In some embodiments, the T-cell is a CD4+ T-cell (a helper T-cell) that recognizes a peptide described herein presented on a class II MHC (e.g., HLA-DP, -DM, -DOA, -DOB, -DQ, and -DR). In certain embodiments, such T cells are prepared by any one of the methods disclosed herein.

In some embodiments, the T cells provided herein can be engineered to express a chimeric antigen receptor (CAR). A wide variety of CAR have been described in the scientific literature. In general CAR include an extracellular antigen-binding domain (e.g., a scFv derived from variable heavy and light chains of an antibody), a spacer domain, a transmembrane domain and an intracellular signaling domain. Accordingly, in some embodiments, CMV-specific T cells (e.g., the CMV peptide epitope-pool stimulated CTLs provided) express a CAR targeting an extracellular molecule (e.g., a tumor antigen such as HER2) associated with disease cells such as cancer cells (e.g., a tumor cell).

In some aspects, provided herein are methods of generating, activating and/or inducing proliferation of T-cells (e.g., CTLs) that recognize one or more of the CMV epitopes described herein. In some embodiments, a sample comprising CTLs (e.g., a PBMC sample) is isolated, exposed to a pool of immunogenic peptides disclosed herein, and the stimulated CTLs harvested. Preferably, the pool of immunogenic peptides consists essentially of each of the CMV peptide epitope amino acid sequences set forth in Table 1. In certain embodiments, the exposed sample is incubated for at least 14 days. In some such embodiments, the exposed sample is incubated with IL-21 on Day 0. Preferably, the exposed sample is incubated with IL-2 on day 2. In more preferred embodiments, incubation of the exposed sample includes addition of IL-2 every three days.

In some embodiments, the PBMC sample is derived from a healthy donor. In certain embodiments, the PBMCs are derived from an immunocompromised donor. In some such embodiments, the donor is undergoing immunosuppressive therapy. In some embodiments, the donor is a solid organ transplant recipient. In further embodiments, the donor is receiving anti-viral therapy.

In some embodiments, a sample comprising CTLs (e.g., a PBMC sample) is incubated in culture with an APC provided herein (e.g., an APCs that present a peptide comprising a CMV epitope described herein on a class I MHC complex). The APCs may be autologous to the subject from whom the T-cells were obtained. In some embodiments, the sample containing T-cells is incubated 2 or more times with APCs provided herein. In some embodiments, the T-cells are incubated with the APCs in the presence of at least one cytokine, e.g., IL-2, IL-4, IL-7, IL-15 and/or IL-21. Exemplary methods for inducing proliferation of T-cells using APCs are provided, for example, in U.S. Pat. Pub. No. 2015/0017723, which is hereby incorporated by reference.

In some aspects, provided herein are compositions (e.g., therapeutic compositions) comprising T-cells (e.g., CMV peptide-specific CTLs provided herein) and/or APCs provided herein. In some embodiments, such compositions are used to treat and/or prevent a CMV infection, reactivation, and/or disease in a subject by administering to the subject an effective amount of the composition. The T-cells and/or APCs may be autologous or not autologous to the subject. In some embodiments, the T-cells and/or APCs are stored in a cell bank before they are administered to the subject. In certain embodiments, the subject may be a solid organ transplant recipient.

### Pharmaceutical Compositions

In some aspects, provided herein is a composition (e.g., a pharmaceutical composition), containing a CTL, or preparation thereof, formulated together with a pharmaceutically acceptable carrier, as well as methods of administering such pharmaceutical compositions.

In some embodiments, the composition may further comprise an adjuvant. As used herein, the term “adjuvant” broadly refers to an immunological or pharmacological agent that modifies or enhances the immunological response to a composition in vitro or in vivo. For example, an adjuvant might increase the presence of an antigen over time, help absorb an antigen-presenting cell antigen, activate macrophages and lymphocytes and support the production of cytokines. By changing an immune response, an adjuvant might permit a smaller dose of the immune interacting agent or preparation to increase the dosage effectiveness or safety. For example, an adjuvant might prevent T-cell exhaustion and thus increase the effectiveness or safety of a particular immune interacting agent or preparation. Examples of adjuvants include, but are not limited to, an immune modulatory protein, Adjuvant 65, α-GalCer, aluminum phosphate, aluminum hydroxide, calcium phosphate, β-Glucan Peptide, CpG DNA, GPI-0100, lipid A and modified versions thereof (e.g., monophosphorylated lipid A, lipopolysaccharide, Lipovant, Montanide, N-acetyl-muramyl-L-alanyl-D-isoglutamine, Pam3CSK4, quil A and trehalose dimycolate.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions of this invention suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents of the present invention, which may be used in a suitable hydrated form, and/or the pharmaceutical compositions of the present invention, are formulated into pharmaceutically-acceptable dosage forms by conventional methods known to those of skill in the art.

### Therapeutic Methods

In certain embodiments, provided herein are methods of treating or preventing CMV infection, reactivation, and/or disease (e.g., end-organ disease in solid organ transplant recipients) in a subject comprising administering to the subject peptide-specific T cells (or a pharmaceutical composition comprising said T cells) prepared according to a method provided herein.

In some embodiments, provided herein is a method of treating or preventing a CMV infection in a subject. In certain embodiments, provided herein is a method of treating or preventing CMV reactivation or a CMV-associated condition in a subject. In preferred embodiments, the method comprises administering to the subject CTLs prepared according to a method provided herein. For example and without limitation, an isolated PBMC sample is exposed to a pool of immunogenic peptides according to a method provided herein. In some such embodiments, the pool of immunogenic peptides induces stimulation and proliferation of CMV peptide-specific T cells. In some embodiments, the CTLs administered to the subject are autologous. In certain embodiments, the infection is a recurrent CMV infection. In some embodiments, the subject treated is immunocompromised. For example, in some embodiments, the subject has a T-cell deficiency. In some embodiments, the subject has leukemia, lymphoma or multiple myeloma. In some embodiments, the subject is infected with HIV and/or has AIDS. In some embodiments, the subject has undergone a tissue, organ and/or bone marrow transplant. In some such embodiments, the subject is the recipient of a solid organ transplant. In some embodiments, the subject is being administered immunosuppressive drugs. In some embodiments, the subject has undergone and/or is undergoing a chemotherapy. In some embodiments, the subject has undergone and/or is undergoing radiation therapy.

In some embodiments, the subject is also administered an anti-viral drug. In some such embodiments, the anti-viral drug is for treating CMV infection (e.g., the anti-viral drug inhibits CMV replication). For example, in some embodiments, the subject is administered ganciclovir, valganciclovir, foscarnet, cidofovir, acyclovir, formivirsen, maribavir, BAY 38-4766 or GW275175X. In certain embodiments, the CMV infection is drug-resistant. For example, in some embodiments the CMV infection is ganciclovir-resistant.

Expression of biomarkers by the CMV peptide-specific T cells may be assessed by any suitable method, such as flow cytometry. In some embodiments, the CMV peptide-specific T cells are stimulated by CMV-specific peptides and sorted via flow cytometry. Preferably, the CMV peptide-specific T cells undergo stimulation and/or surface staining according to the protocols exemplified in Examples 1, 4, 5, or any combination thereof. In some embodiments, the CMV peptide-specific T cells are incubated with one or more antibodies specific for CD107a, and subsequently sorted by flow cytometry. In some embodiments, the CMV peptide-specific T cells are incubated with one or more antibodies that bind to intracellular cytokines, such as antibodies specific for IFNγ, IL-2, and/or TNF. In some embodiments, the CMV peptide-specific T cells are incubated with antibodies for intracellular cytokines and subsequently sorted via flow cytometry.

In some aspects, provided herein are methods of selecting a subject for adoptive immunotherapy by obtaining a PMBC sample from the subject, isolating the autologous T cells, determining the CMV reactivity of the autologous T cells, and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70% or 80% of the autologous T cells are CMV reactive, selecting the subject for adoptive immunotherapy.

In some aspects, provided herein are methods of selecting a subject for adoptive immunotherapy by obtaining a sample comprising T cells (e.g., CTLs) from the subject, isolating the autologous T cells, and determining the CD107a expression of the autologous T cells, and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70% or 80% of the autologous T cells express CD107a, selecting the subject for adoptive immunotherapy.

In some aspects, provided herein are methods of selecting a subject for adoptive immunotherapy by obtaining a sample comprising T cells (e.g., CTLs) from the subject, isolating the autologous T cells, determining the IFNγ expression of the autologous T cells, and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70% or 80% of the autologous T cells express IFNγ selecting the subject for adoptive immunotherapy.

In some aspects, provided herein are methods of selecting a subject for adoptive immunotherapy by obtaining a sample comprising T cells (e.g., CTLs) from the subject, isolating the autologous T cells, determining the TNF expression of the autologous T cells, and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70% or 80% of the autologous T cells express TNF, selecting the subject for adoptive immunotherapy.

In some aspects, provided herein are methods of selecting a subject for adoptive immunotherapy by obtaining a sample comprising T cells (e.g., CTLs) from the subject, isolating the autologous T cells, determining the IL-2 expression of the autologous T cells, and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70% or 80% of the autologous T cells express 11-2, selecting the subject for adoptive immunotherapy.

In some embodiments, the methods further comprise obtaining a sample comprising the T cells from the subject (e.g., obtaining a PBMC sample from the subject). In some embodiments, the autologous T cells (e.g., CD4+ T cells or CD8+ T cells) are isolated form the sample. In some embodiments, the sample is comprised mostly or completely of autologous T cells.

Provided herein are methods of treating or preventing CMV infection in a subject, comprising administering to the subject immunogenic peptide pool-stimulated T cells (e.g., autologous CMV peptide-specific CTLs) expressing T cell receptors that specifically bind to one or more CMV peptides presented on a class I and/or class II MHC, (e.g. any one of the peptides set forth in Table 1 or combination thereof). In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70%, 80% or 90% of the T cells (e.g., CTLs) in the sample express CD107a. In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70%, 80% or 90% of the T cells (e.g., CTLs) in the sample express IFNγ. In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70%, 80% or 90% of the T cells (e.g., CTLs) in the sample express TNF. In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70%, 80% or 90% of the T cells (e.g., CTLs) in the sample express IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express CD107a and IFNγ.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express CD107a and TNF.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express CD107a and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express IFNγ and TNF.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express IFNγ and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express TNF and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express IFNγ, TNF, and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express CD107a, TNF, and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express CD107a, IFNγ, and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express CD107a, IFNγ, and TNF.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) in the sample express CD107a, IFNγ, TNF, and IL-2.

In some embodiments of the methods disclosed herein, the T cells (e.g., CTLs) display reactivity against multiple peptide epitopes derived from multiple CMV antigens. 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T cells (e.g., CTLs) are reactive to more than one CMV epitope. In certain embodiments, the T cells (e.g., CTLs) are reactive to any one of the CMV peptide epitope amino acid sequences set forth in Table 1, or combinations thereof. In some embodiments, the T cells (e.g., CTLs) are reactive to any one of pp50, pp65, IE-1, gB, gH, or combinations thereof.

T cell biomarker expression and/or CMV reactivity may be measured and/or analyzed either before or after T cell (e.g., CTL) expansion by any one of the methods disclosed herein, e.g., by exposure to a pool of immunogenic CMV peptide epitopes.

In some embodiments, CMV reactivity and biomarker expression is quantified prior to stimulation of the T cells (e.g., CTLs). Alternatively or additionally, CMV reactivity and biomarker expression may be quantified after stimulation of the T cells (e.g., CTLs) In some embodiments, CMV reactivity is measured by quantifying the percentage of T cells in the sample that express CD107a. In some embodiments, CMV reactivity is measured by quantifying the percentage of T cells in the sample that express IFNγ. In some embodiments, CMV reactivity is measured by quantifying the percentage of T cells in the sample that express TNF. In some embodiments, CMV reactivity is measured by quantifying the percentage of T cells in a sample that express IL-2. In some embodiments, CMV reactivity is measured as a percentage of T cells that express multiple biomarkers (e.g., two or more of CD107a, IFNγ, TNF, and IL-2, preferably all four). In some embodiments, the CMV reactivity is calculated by quantifying the percentage of autologous T cells in a sample that express CD107a, IFNγ, TNF, and IL-2. T cells may be isolated from a sample (e.g., a PBMC sample or a sample comprising T cells) either before or after CMV reactivity percentage quantification. Therefore, in some embodiments, CMV reactivity is the percentage of T cells having the desired characteristic(s) in a sample that comprises mostly T cells.

In some embodiments, CMV reactivity is measured by quantifying the percentage of CD8+ lymphocytes in the sample that express CD107a. In some embodiments, CMV reactivity is measured by quantifying the percentage of CD8+ lymphocytes in the sample that express IFNγ. In some embodiments, CMV reactivity is measured by quantifying the percentage of CD8+ lymphocytes in the sample that express TNF. In some embodiments, CMV reactivity is measured by quantifying the percentage of CD8+ lymphocytes in a sample that express IL-2. In some embodiments, CMV reactivity is measured as a percentage of CD8+ lymphocytes that express multiple biomarkers (e.g., two or more of CD107a, IFNγ, TNF, and IL-2, preferably all four). CD8+ lymphocytes may be isolated from a sample (e.g., a PBMC sample or a sample of CD8+ lymphocytes) either before or after CMV reactivity percentage quantification. Therefore, in some embodiments, CMV reactivity is the percentage of CD8+ lymphocytes having the desired characteristic(s) in a sample that comprises mostly or CD8+ lymphocytes.

In some embodiments, CMV reactivity is measured by quantifying the percentage of CD3+ lymphocytes in the sample that express CD107a. In some embodiments, CMV reactivity is measured by quantifying the percentage of CD3+ lymphocytes in the sample that express IFNγ. In some embodiments, CMV reactivity is measured by quantifying the percentage of CD3+ lymphocytes in the sample that express TNF. In some embodiments, CMV reactivity is measured by quantifying the percentage of CD3+ lymphocytes in a sample that express IL-2. In some embodiments, CMV reactivity is measured as a percentage of CD3+ lymphocytes that express multiple biomarkers (e.g., two or more of CD107a, IFNγ, TNF, and IL-2, preferably all four). CD3+ lymphocytes may be isolated from a sample (e.g., a PBMC sample or a sample of CD3+ lymphocytes) either before or after CMV reactivity percentage quantification. Therefore, in some embodiments, CMV reactivity is the percentage of CD3+ lymphocytes having the desired characteristic(s) in a sample that comprises mostly CD3+ lymphocytes.

In some embodiments, the method further comprises analyzing the expression of CD107a, IFNγ, TNF, or IL-2 by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express CD107a, IFNγ, TNF, or IL-2, administering the CMV peptide-specific autologous T cells (e.g., CTLs) to the subject.

In some embodiments, the method further comprises analyzing the expression of multiple biomarkers by the CMV peptide-specific T cells (e.g., CTLs), and, if at least two biomarkers are expressed by the CMV peptide-specific T cells, administering the CMV peptide-specific T cells to the subject. In some such embodiments, the method further comprises analyzing the expression of CD107a and TNF by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express CD107a and TNF, administering the peptide-specific autologous T cells (e.g., CTLs) to the subject.

In some embodiments, the method further comprises analyzing the expression of CD107a and IFNγ by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express CD107a and IFNγ, administering the CMV peptide-specific T cells (e.g., CTLs) to the subject.

In some embodiments, the method further comprises analyzing the expression of CD107a and IL-2 by the proliferated peptide-specific autologous T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express CD107a and IL-2, administering the CMV peptide-specific T cells (e.g., CTLs) to the subject.

In some embodiments, the method further comprises analyzing the expression of TNF and IL-2 by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express TNF and IL-2, administering the CMV peptide-specific T cells (e.g., CTLs) to the subject.

In some embodiments, the method further comprises analyzing the expression of IFNγ and IL-2 by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific autologous T cells (e.g., CTLs) express IFNγ and IL-2, administering the CMV peptide-specific T cells (e.g., CTLs) to the subject.

In some embodiments, the method further comprises analyzing the expression of IFNγ and TNF by the proliferated CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express IFNγ and TNF, administering the CMV peptide-specific T cells (e.g., CTLs) to the subject.

In some embodiments, the method further comprises analyzing the expression of CD107a, IFNγ, and TNF by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express CD107a, IFNγ, and TNF, administering the CMV peptide-specific T cells (e.g., CTLs) to the subject.

In some embodiments, the method further comprises analyzing the expression of CD107a, IFNγ, and IL-2 by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express CD107a, IFNγ, and IL-2, administering the CMV peptide-specific T cells (e.g., CTLs) to the subject.

In some embodiments, the method further comprises analyzing the expression of CD107a, IL-2, and TNF by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express CD107a, IL-2, and TNF, administering the peptide-specific T cells (e.g., CTLs) to the subject.

In some embodiments, the method further comprises analyzing the expression of IFNγ, IL-2, and TNF by the CMV peptide-specific T cells (e.g., CTLs), and if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific T cells (e.g., CTLs) express IFNγ, IL-2, and TNF, administering the CMV peptide-specific T cells (e.g., CTLs) to the subject.

In some embodiments, if at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the CMV peptide-specific autologous T cells (e.g., CTLs) express CD107a, IFNγ, TNF, and IL-2, the autologous T cells (e.g., CTLs) are administered to the subject.

The CMV peptide-specific autologous T cells (e.g., CTLs) may have at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% CMV reactivity.

In some embodiments, the method further comprises analyzing the CMV reactivity of the CMV peptide-specific T cells (e.g., CTLs), and, if the reactivity is to more than one epitope and at least a threshold percentage (e.g., at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90%) of the CMV peptide-specific T cells (e.g., CTLs) are CMV reactive, administering the CMV peptide-specific T cells (e.g., CTLs) to the subject.

In some embodiments, about 1×10⁵ to about 1×10⁸ T-cells are administered to the subject per dose of T cells. In some embodiments, about 1×10⁶ to about 1×10⁷T cells are administered to the subject per dose of T cells. In some embodiments, 5×10⁶, 1×10⁷, 1.5×10⁷, or 2×10⁷ T cells (e.g., CTLs) are administered to the subject. Multiple doses may be administered to the subject. In some embodiments, an initial dose of T cells (e.g., autologous CTLs) is administered, and one or more additional doses of T cells (e.g., autologous CTLs) are administered, e.g., at increasing doses along the course of therapy. In some embodiments, two or more, three or more, four or more, five or more, six or more, seven or more, eight or more, nine or more, or ten or more doses are administered. The subject may be administered additional doses that are the same or different from the initial dose. For example, a lower dose may be administered followed by a higher dose. The doses may be administered daily, twice a week, weekly, biweekly, once a month, once every two months, once every three months, or once every six months. In some embodiments, the subject does not experience any adverse effects as a result of T cell (e.g., autologous CTL) administration.

In some aspects, the method further comprises assessing the efficacy of adoptive immunotherapy by measuring the CMV viral load in a subject with CMV infection, reactivation, or associated disease. In some embodiments, the subject has received a solid organ transplant. By way of non-limiting example, CMV viral load may be measured by obtaining a first sample (e.g. a blood sample) from the subject, assessing the viral load in the first sample using methods known in the art (preferably before a CTL administration) and, after a period of time, obtaining a second sample from the subject (preferably after a CTL administration), assessing the viral load in the second sample, and if the viral load in the second sample is less than the first sample, the CMV infection, reactivation, or associated disease has improved and/or not progressed. Additional samples may be obtained and compared to previous samples. Also provided herein are methods of reducing the viral load in a subject with CMV infection, reactivation, or associated disease by administering to the subject immunogenic peptide pool-stimulated T cells (e.g., the CMV peptide-specific autologous CTLs disclosed herein). A change (e.g., reduction) in viral load may be measured by using methods known in the art, such as nucleic acid-based assays (e.g. nucleic acid tests (NATs) and nucleic acid amplification tests (NAATs)) or non-nucleic acid tests (e.g., quantitative enzyme immunoassays). Viral load may be reduced by about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, 90%, 91%, 92%, 93%, 94%, 95%, 96%, 97%, 98%, 99%, or 100% following administration of T cells.

In some embodiments, the methods comprise improving or stabilizing a symptom or condition of a subject with CMV infection, reactivation, or associated disease, by administering to the subject immunogenic peptide pool-stimulated T cells (e.g., CTLs, such as the CMV peptide specific autologous CTLs described herein). Also provided herein are methods of reducing or resolving DNAemia; and/or reducing, stabilizing, or ceasing CMV-associated end organ disease in a subject infected with CMV, comprising administering to the subject immunogenic peptide pool-stimulated T cells (e.g., CTLs, such as the CMV peptide-specific autologous CTLs described herein). In some embodiments, provided herein are methods of reducing or ceasing the use of anti-viral therapy infected with CMV, comprising administering to the subject immunogenic peptide pool-stimulated T cells (e.g., CTLs, such as the CMV peptide-specific autologous CTLs described herein). In preferred embodiments, the subject has received a solid organ transplant. In more preferred embodiments, the subject is suffering from a ganciclovir-resistant CMV infection, reactivation, or associated disease.

In some embodiments, the subject has cancer. In some embodiments, the methods described herein may be used to treat any cancerous or pre-cancerous tumor. In some embodiments, the cancer expresses one or more of the CMV epitopes provided herein (e.g., the CMV epitopes listed in Table 1). In some embodiments, the cancer includes a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; glioblastoma multiforme (GBM); oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the subject is also administered an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn0), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®). In some embodiments, the subject is also administered a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

In some embodiments, the subject is also administered an immunotherapeutic agent. Immunotherapy refers to a treatment that uses a subject's immune system to treat or prevent a condition, e.g. cancer vaccines, cytokines, use of target-specific antibodies, T-cell therapy, and dendritic cell therapy.

In some embodiments, the subject is also administered an immune modulatory protein. Examples of immune modulatory proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophase inflammatory protein-1-delta (“MIP-1 delta”), Platelet-derived growth factor subunit B (“PDGF-BB”), Chemokine (C-C motif) ligand 5, Regulated on Activation, Normal T-cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-alpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κ B (“RANK”).

In some embodiments, the subject is also administered an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen-binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In some embodiments, a composition provided herein (e.g., a vaccine composition provided herein) is administered prophylactically to prevent cancer and/or a CMV infection. In some embodiments, the vaccine is administered to inhibit tumor cell expansion. The vaccine may be administered prior to or after the detection of cancer cells or CMV infected cells in a patient. Inhibition of tumor cell expansion is understood to refer to preventing, stopping, slowing the growth, or killing of tumor cells. In some embodiments, after administration of a vaccine comprising peptides, nucleic acids, antibodies or APCs described herein, a proinflammatory response is induced. The proinflammatory immune response comprises production of proinflammatory cytokines and/or chemokines, for example, interferon gamma (IFN-γ) and/or interleukin 2 (IL-2). Proinflammatory cytokines and chemokines are well known in the art.

Conjoint therapy includes sequential, simultaneous and separate, and/or co-administration of the active compounds in such a way that the therapeutic effects of the first agent administered have not entirely disappeared when the subsequent treatment is administered. In some embodiments, the second agent may be co-formulated with the first agent or be formulated in a separate pharmaceutical composition.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In some embodiments, the methods provided herein further comprise treating the identified subject using a therapeutic method provided herein (e.g., by administering to the subject a pharmaceutical composition provided herein).

### EXAMPLES

### Example 1: Patient Characteristics

In order to assess the safety of autologous T-cell therapy in solid organ transplant (SOT) recipients with CMV-associated complications, patients were selected and deemed eligible once they had met one of the four following criteria:

(A) CMV reactivation or disease (as defined by histology) following successful initial therapy, e.g., ganciclovir-resistant CMV reactivation;

(B) Persistent CMV disease, i.e. no response to 2 weeks of salvage foscarnet or other second-line anti-viral agent, e.g., recurrent CMV recrudescence due to refractoriness to second-line drug therapy;

(C) Persistent CMV replication (more than 6 weeks by PCR) despite appropriate anti-viral therapy; or

(D) Any CMV reactivation or disease where anti-viral therapy is contraindicated on the basis of intolerance or end organ limitation (e.g. renal impairment, marrow dysfunction), e.g., end-organ CMV disease or intolerance to anti-viral drug therapy.

Anti-viral drug therapy was administered as per the institutional guidelines. Patients received up to six doses of in vitro expanded T-cells at 1-2×10⁷ cells/m² every two weeks. Each participant was monitored for safety, clinical symptoms, viral load and immune reconstitution for 28 weeks after the completion of adoptive T-cell therapy. Viral load monitoring was undertaken using an in-house qualitative assay as described previously (Hill et al. 2016 Am J Transplant 2010; 10(1): 173-9).

Results

The clinical characteristics of the participants included in this study are provided in Table 2. In total, 21 SOT recipients (13 renal, 8 lung, 1 heart) were included in the study. Two of the lung transplant patients included in the follow-up analyses were previously treated under the Special Access Scheme of the Therapeutic Goods Administration (Holmes-Liew et al. Clinical & translational immunology 2015; 4(3): e35; Pierucci et al. J Heart Lung Transplant 2016; 35(5): 685-7). Of the 21 patients analyzed, 13 SOT recipients were allocated to intervention and received a maximum of six doses of adoptive T-cell therapy. One patient discontinued therapy after a single dose and no immune monitoring was performed. Of the remaining eight patients, seven did not receive adoptive T-cell therapy due to improvement in their clinical status, and therapy could not be prepared for one patient.

TABLE 2
Clinical Profile of SOT Recipients Enrolled in Study

| | | | | | | | | Donor/ |
|---|---|---|---|---|---|---|---|---|
| | | | | | Anti- | | CMV | Recipient |
| Patient | | | Criteria for | Immuno- | Viral | Drug | Disease | CMV |
| Code | Age/Sex | Organ | Recruitment | suppression | Treatment | Resistance | History | Status |
| 1553PAH01 | 61M | Kidney | B, C | TAC; | GCV; | Nil | Stomach, | +/− |
| | | | | MMF; | FOS | | lung, colon | |
| | | | | MePRD | | | | |
| 1553PAH02 | 45F | Kidney | A | TAC; | VGCV | Nil | Colon | +/+ |
| | | | | MMF; | | | | |
| | | | | PRD | | | | |
| 1553PAH03 | 57M | Kidney | A | CSA; PRD | VGCV; | Nil | Not | Unk/+ |
| | | | | | GCV | | detected | |
| 1553PAH04 | 64F | Kidney | A | TAC; | VGCV | Nil | Colon | +/+ |
| | | | | MMF; | | | | |
| | | | | PRD | | | | |
| 1553PAH05 | 23M | Kidney | C | TAC; | VGCV; | Nil | Colitis, | +/+ |
| | | | | MMF; | GCV; | | pneumonitis | |
| | | | | PRD | FOS; LEF | | | |
| 1553PAH06 | 57M | Kidney | A | TAC; | VGCV; | GCV | Colitis | −/− |
| | | | | MMF; | GCV | | | |
| | | | | PRD | | | | |
| 1553PAH07 | 26F | Kidney | A | TAC; | VGCV; | N.A. | Colitis | +/+ |
| | | | | MMF; | GCV | | | |
| | | | | PRD | | | | |
| 1553PAH08 | 26M | Kidney | B, C | TAC; | VGCV; | Nil | Not | +/− |
| | | | | MMF; | GCV; | | detected | |
| | | | | PRD | FOS | | | |
| 1553PAH09 | 44M | Kidney | C | TAC; | VGCV; | Nil | Not | +/− |
| | | | | MMF; | GCV | | detected | |
| | | | | PRD; | | | | |
| | | | | MePRD | | | | |
| 1553PAH10 | 53F | Kidney | A | TAC; | VGCV; | Nil | Not | +/+ |
| | | | | MMF; | GCV | | detected | |
| | | | | PRD | | | | |
| 1553PAH11 | 45M | Kidney | C | TAC; | VGCV; | Nil | Not | +/− |
| | | | | MMF; | GCV | | detected | |
| | | | | PRD | | | | |
| 1553PAH12 | 43F | Kidney | C | TAC; | VGCV; | Nil | Not | +/− |
| | | | | MMF; | GCV | | detected | |
| | | | | PRD | | | | |
| 1553PAH13 | 53M | Kidney | A | TAC; | VGCV; | Nil | Not | +/+ |
| | | | | MMF; | GCV | | detected | |
| | | | | PRD | | | | |
| 1553PCH01 | 62M | Lung | B | EVR, PRD | GCV; | GCV | Oesophagitis | −/− |
| | | | | | FOS | | | |
| 1553PCH02 | 55M | Lung | A | TAC; | VGCV; | GCV | Colitis | +/+ |
| | | | | MMF; | GCV; | | | |
| | | | | EVR; | FOS; | | | |
| | | | | AZA; PRD | IVIG | | | |
| 1553PCH03 | 62F | Lung | C | TAC; | VGCV; | Nil | Pneumonitis | +/− |
| | | | | MMF; | GCV; | | | |
| | | | | EVR; | FOS | | | |
| | | | | AZA; | | | | |
| | | | | MYF | | | | |
| 1553PCH04 | 29F | Lung | A | CSA; | VGCV: | GCV | Pneumonitis, colitis | +/− |
| | | | | TAC; | GCV; | | | |
| | | | | MMF; | FOS; | | | |
| | | | | EVR | IVIG; | | | |
| | | | | | LEF | | | |
| 1553PCH05 | 66M | Lung | A | CSA; | VGCV; | Nil | Colitis, | +/− |
| | | | | TAC; | GCV | | mouth ulcer | |
| | | | | MMF; | | | | |
| | | | | AZA | | | | |
| 1553RAH01 | 64M | Lung | D | TAC; | VGCV; | N.A. | Lung | +/− |
| | | | | PRD | GCV | | | |
| SASRAH01 | 41F | Lung | A, B | TAC; | VGCV; | GCV | Hepatitis, | +/− |
| | | | | PRD; | GCV; | ULP7 | lung | |
| | | | | AZA; | FOS | L595S | | |
| | | | | EVR; LEF; | | | | |
| | | | | MePRD | | | | |
| SASSVH01 | 56M | Lung | A, B | N.A. | VGCV: | GCV; | Not | +/− |
| | | | | | GCV; | L595S; | detected | |
| | | | | | FOS; | FOS; | | |
| | | | | | CDV | UL54; | | |
| | | | | | | L415N; | | |
| | | | | | | S734P; | | |
| | | | | | | I840T | | |
| 1553PCH06 | 61M | Heart | D | CSA; | VGCV | Nil | Nil | +/+ |
| | | | | MMF | | | | |

N.A. Not available
A: CMV reactivation or disease (as defined by histology) following successful initial therapy.
B: Persistent CMV disease, i.e. no response to 2 weeks of salvage foscamet or other second line anti-viral agent.
C: Persistent CMV replication (more than 6 weeks by PCR) despite appropriate anti-viral therapy.
D: Any CMV reactivation or disease where anti-viral therapy is contraindicated on the basis of intolerance or end organ limitation (e.g. renal impairment, marrow dysfunction).
AZA: Azathioprine;
CSA: Cyclosporin;
EVR: Everolimus;
LEF: Leflunomide;
MePRD: Methylprednisolone;
MMF: Mycophenolate;
PRD: Prednisolone;
TAC: Tacrolimus.
CDV: Cidofovir;
FOS: Foscarnet;
GCV: Gancyclovir;
VGCV: Valgancyclovir.

### Example 2: T-Cell Therapy Preparation

To generate the CMV-specific T-cell therapy, peripheral blood mononuclear cells (PBMCs) acquired from each patient were each stimulated with a clinical-grade CMV peptide pool that included pre-defined HLA class I and class II-restricted peptide epitopes from pp65, pp50, IE-1, gH and gB (Table 1), in the presence of IL-21 (40 ng/mL on Day 0). The stimulated samples were then cultured in Grex-10 culture flasks (Wilson Wolf Corporation, Saint Paul, Minn.) at a starting cell density of 2-5×106 cells/cm2. These cultures were supplemented with IL-2 (120 IU/mL) on Day 2 and every three days thereafter. On Day 14, expanded T-cells were harvested and frozen in 1 mL single-dose aliquots in Albumex 4 (CSL Behring, Broadmeadows, Australia) containing 10% dimethyl sulfoxide (WAK-Chemie Medical GmbH, Steinbach, Germany). The T-cells were tested for microbial contamination prior to infusion, and were phenotypically and functionally characterised using Multitest 6-Colour TBNK Reagent (BD Biosciences, San Jose, Calif.) and intracellular cytokine staining (detailed below). For adoptive transfer, T-cells were thawed into 19 mL clinical grade normal saline and infused intravenously over a period of 5-10 min.

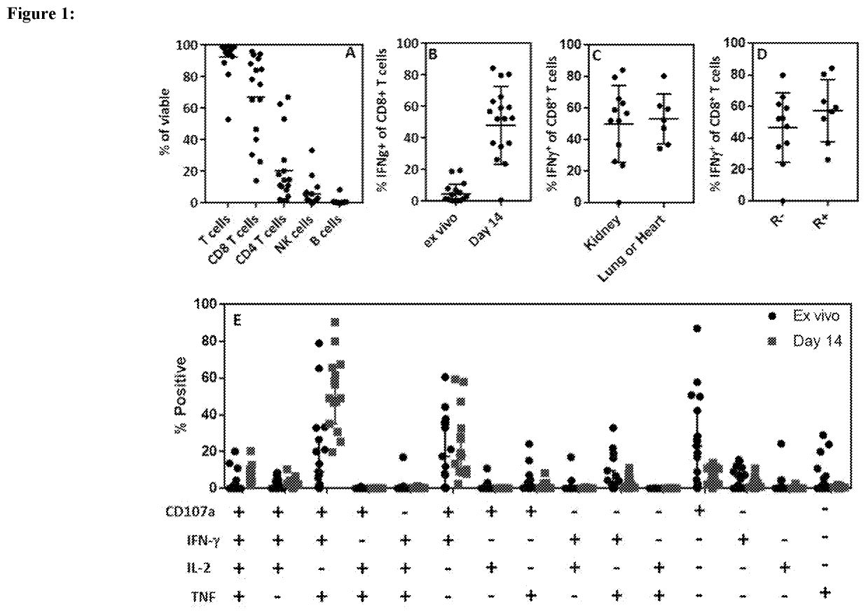

Results CMV-specific T-cells were successfully expanded from 20 of the 21 patients, and their antigen specificity was assessed by intracellular IFN-γ analysis (Table 3). The CMV peptide pool-expanded cells were predominantly CD3+CD8+ T-cells (FIG. 1A), with a median specificity of 51.2% (FIG. 1B). The frequency of IFN-γ-producing CD8+ T-cells did not differ significantly between kidney and lung/heart transplant recipients (FIG. 1C) or pre-transplant CMV seropositive and CMV seronegative individuals (FIG. 1D). A marked improvement in the polyfunctionality of the CMV-specific T-cells was observed following in vitro expansion, with an increase in the proportion of cells capable of producing IFN-γ, TNF and CD107a (FIG. 1E). T-cells generated from the majority of the patients showed reactivity against multiple peptide epitopes encoded by multiple CMV antigens (Table 3).

TABLE 3
CMV-specifIc reactivity of in vitro-expanded T-cells from SOT recipients

| | | | CMV-Specific T-cell | | |
|---|---|---|---|---|---|
| | Organ | Organ | Response# | | |
| | Recipient | Donor | Ex vivo (prior | Day | |
| Patient Code | HLA Type | HLA Type | to stimulation) | 14 | CMV Epitopes Targeted |
| 1553PAH01 | A1 A11 B8 | A31 A33 | 0.24 | 0.0 | N.A. |
| | B60 | B51 B58 | | | |
| 1553PAH02 | A2 A34 B44 | A1 A2 B44 | 5.15 | 79.9 | NLV (pp65, A2); VLE/YIL |
| | B75 | B44 | | | (IE-1, A2) DEL (IE-1, B44) |
| 1553PAH04 | A2 A25 B7 | A2 A24 B7 | 0.43 | 47.6 | RPH (pp65, B7); TPR (pp65, |
| | B35 | B62 | | | B7) |
| 1553PAH05 | A24 A34 B56 | A3 A31 | 0.05 | 24.3 | QYD (pp65, A24) |
| | Cw1 Cw7 | B51 B7 | | | |
| 1553PAH06 | A2 A32 B7 | A2 A11 | 17.67 | 77.2 | NLV (pp65, A2); RPH (pp65, |
| | B27 | B13 B46 | | | B7); TPR (pp65, B7) |
| 1553PAH07 | A2 A2 B44 | A2 A2 B7 | 0 | 36.5 | NLV (pp65, A2) |
| | B51 | B44 | | | |
| 1553PAH08 | A1 A29 B8 | A1 A2 B44 | 0 | 22.9 | VTE (pp50, A1); ELR/K (IE-1, |
| | B52 | B57 | | | B8); |
| 1553PAH09 | A3 A29 B44 | A2 A3 B7 | 0.09 | 48.4 | TRA (pp65, Cw6) |
| | B45 Cw6 | B51 | | | |
| | Cw16 | | | | |
| 1553PAH10 | A11 A24 B7 | A2 A31 | 3.14 | 66.0 | RPH (pp65, B7); TPR (pp65, |
| | B55 Cw7 | B62 B60 | | | B7); QYD (pp65, A24); AYA |
| | Cw7 | | | | (IE-1, A24) |
| 1553PAH11 | A3 A24 B35 | A2 A23 | 3.21 | 59.1 | IPS (pp65, B35); AYA (IE-1, |
| | B60 | B44 B62 | | | A24) |
| 1553PAH12 | A25 A68 B8 | A1 A11 B8 | 0.44 | 61.6 | IPS (pp65, B35); ELR/K (IE-1, |
| | B35 | B35 | | | B8) |
| 1553PAH13 | A2 A11 B35 | A11 A32 | 3.21 | 60.2 | NLV (pp65, A2); IPS (pp65, |
| | B35 Cw4 | B58 B62 | | | B35) |
| | Cw4 | Cw4 Cw7 | | | |
| 1553PCH01* | A3 A31 B38 | A2 A3 B7 | 0.00 | 56.9 | KAR (IE-1; A31) |
| | B65 Cw8 | B65 | | | |
| 1553PCH02 | A1 A3 B42 | A2 A3 B7 | 0.87 | 57.3 | TRA (pp65, Cw6); VTE (pp50, |
| | B57 Cw17 | B62 | | | A1) |
| 1553PCH03 | A1 A3 B7 B8 | A1 A2 B51 | 8.74 | 48.0 | RPH (pp65, B7); TPR (pp65, |
| | Cw7 Cw7 | B57 | | | B7); YSE (pp65, A1); VTE |
| | | | | | (pp50; A1); QIK (IE-1; B8); |
| | | | | | CRV (IE-1; Cw7) |
| 1553PCH04 | A2 A11 B44 | A32 A62 | 6.35 | 63.6 | TRA (pp65, Cw6) |
| | B50 Cw5 | B44 B53 | | | |
| | Cw6 | | | | |
| 1553PCH05 | A2 A3 B27 | A3 A29 | 1.32 | 26.9 | NLV (pp65, A2) |
| | B49 Cw1 | B50 B51 | | | |
| | Cw7 | | | | |
| 1553RAH01 | A2 A23 B44 | N.A. | 0.00 | 31.9 | N.A. |
| | B44 | | | | |
| SASRAH01** | A1 A11 B7 | N.A. | 0.73 | 11.68 | RPH (pp65, B7); TPR (pp65, |
| | B35 Cw4 | | | | B7); YSE (pp65, A1); VTE |
| | Cw7 | | | | (pp50, A1); IPS (pp65, B35); |
| SASSVH01** | A1 A3 B7 B8 | N.A. | 14.22 | 43.94 | RPH (pp65, B7); TPR (pp65, |
| | Cw7 Cw7 | | | | B7); VTE (pp50; A1); ELR |
| | | | | | (IE-1; B8); QIK (IE-1; B8); |
| 1553PCH06 | A2 A24 B44 | A1 A3 B7 | 17.13 | 71.4 | NLV (pp65, A2); VLE/YIL |
| | B56 Cw1 | B8 | | | (IE-1, A2) |
| | Cw5 | | | | |

N.A. Not available
#CMV responses were determined as the proportion of CD8+ T-cells producing IFN-γ
*The KAR peptide was added to the CMV peptide pool for stimulation
**HLA-specific peptide pools were generated to manufacture T-cells for these patients

### Example 3: Clinical Outcomes Following Adoptive Immunotherapy

None of the patients who received adoptive CMV-specific T-cell therapy showed treatment-related grade 3, 4 or 5 adverse events (Table 4). All adverse events that were deemed at least possibly attributable to T-cell infusion were grade 1 and 2, and included fatigue and malaise. Importantly, no adverse events associated with a change in the graft status were detected. Clinical follow-up of patients allocated to T-cell therapy intervention indicated that 11 of the 13 patients showed objective improvement in their symptoms. These included reduction or resolution of CMV reactivation and/or disease and improved response to anti-viral drug therapy. The median peak viral load prior to adoptive T-cell therapy in the 11 patients who showed a clinical response was 3.2×104 CMV copies/mL of blood (range 1.4×103-3.44×105 copies). Following adoptive immunotherapy, the median viral load dropped to 1.2×103 CMV copies/mL of blood (range 0-7.9×103 copies; Table 4). Furthermore, many of these patients showed resolution of CMV disease symptoms (Table 4). More importantly, following the completion of adoptive T-cell therapy, the use of anti-viral drug therapy was either completely stopped (5/11) or significantly reduced (6/11; Table 5).

Results

In a cohort of patients (recruited due to evidence of drug resistance/intolerance, persistent viral reactivation or associated disease), no evidence of severe adverse events or any negative impact on the graft following T-cell administration was demonstrated (see Table 4).

TABLE 4
Safety assessment after T-cell therapy

| Adverse events* | No. incidents |
|---|---|
| Grade 1 - Mild | |
| Nausea | 2 |
| Malaise | 2 |
| Fatigue | 2 |
| Altered taste sensation | 2 |
| Grade 2 - Moderate | |
| Fatigue | 1 |
| Halitosis | 1 |
| Microangiopathic haemolytic anaemia | 1 |

*Events possibly or probably related to the T-cell therapy. No adverse events were deemed to be definitely related to the T-cell therapy.

TABLE 5
Clinical responses following adoptive T-cell therapy

| | | | Total | Peak | Peak | Anti- | Anti- | Clinical |
|---|---|---|---|---|---|---|---|---|
| | | | T- | Load | Load | Viral | Viral | Symptoms/ |
| | | | cell | Pre- | Post- | Therapy | Therapy | Management |
| Patient | | No. of | Dose | Infusion | Infusion | Pre-T-cell | Post-T-cell | Post-T-cell |
| Code | Organ | Infusions | (×10⁶) | (×10³) | (×10³) | Infusion | Therapy | Therapy |
| 1553PAH05 | Kidney | 1 | 45.25 | 1.4 | 0.32 | VGCV; | FOS; | DNAemia and |
| | | | | | | GCV; | LEF | CMV disease |
| | | | | | | FOS; | | symptoms |
| | | | | | | LEF | | resolved |
| 1553PAH06 | Kidney | 6 | 245 | 12 | 0.78 | VGCV; | Nil | CMV disease |
| | | | | | | GCV | | symptoms |
| | | | | | | | | resolved |
| 1553PAH08 | Kidney | 5 | 226 | 54 | 7.9 | VGCV; | VGCV; | CMV disease |
| | | | | | | GCV; | IVIG | symptoms |
| | | | | | | FOS | | resolved |
| 1553PAH09 | Kidney | 5 | 180 | 10 | 1.4 | VGCV; | VGCV | Diarrhoea |
| | | | | | | GCV | | resolved; |
| | | | | | | | | immunosuppression |
| | | | | | | | | reduced |
| 1553PCH01 | Lung | 6 | 210 | 8 | 0.12 | GCV; | Nil | FOS stopped |
| | | | | | | FOS | | without viral |
| | | | | | | | | reactivation |
| 1553PCH02 | Lung | 3 | 108 | 48 | 2.3 | VGCV; | Nil | Reduction in |
| | | | | | | GCV; | | DNAemia |
| | | | | | | FOS; | | |
| | | | | | | IVIG | | |
| 1553PCH03 | Lung | 2 | 42 | 12 | 45 | VGCV; | GCV | Died of multi- |
| | | | | | | GCV; | | organ failure |
| | | | | | | FOS | | |
| 1553PCH04 | Lung | 6 | 168 | 17 | 2.9 | VGCV; | IVIG; | Reduction in |
| | | | | | | GCV; | LEF | DNAemia |
| | | | | | | FOS; | | |
| | | | | | | IVIG; | | |
| | | | | | | LEF | | |
| 1553PCH05 | Lung | 6 | 241 | 47 | 0 | VGCV; | VGCV | Reduction in |
| | | | | | | GCV | | DNAemia |
| 1553RAH01 | Lung | 3 | 104 | 18.9 | 17.6 | VGCV; | GCV; | Ongoing elevated |
| | | | | | | GCV | FOS; | CMV PCR, |
| | | | | | | | IVIG | however no end- |
| | | | | | | | | organ disease |
| SASRAH01 | Lung | 4 | 120 | 344 | 1 | VGCV; | Nil | Drug-independent |
| | | | | | | GCV; | | reduction of |
| | | | | | | FOS | | DNAemia |
| SASSVH01* | Lung | 2 | 38.7 (cycle 1) | 95.4 | 2.5 | VGCV; | CDV | Reduction in |
| | | 1 | 22.2 (cycle 2) | | | GCV; | | DNAemia |
| | | | | | | FOS; | | |
| | | | | | | CDV | | |
| 1553PCH06 | Heart | 6 | 204 | 1.5 | 0 | VGCV | Nil | VGCV ceased |
| | | | | | | | | after T-cell |
| | | | | | | | | therapy |

CDV: Cidofovir;
FOS: Foscarnet;
GCV: Gancyclovir;
IVIG: Intravenous CMV immunoglobulin;
LEF: Leflunomide;
VGCV: Valgancyclovir

To assess the impact of adoptive T-cell therapy on CMV-specific T-cell immune reconstitution, a longitudinal intracellular cytokine analysis following the immunotherapy was conducted, and overlaid with virological monitoring in each patient. Briefly, To characterize the T-cell therapy and the PBMCs isolated from follow-up blood samples, cells were stimulated with CMV peptide epitopes and assessed for the expression of IFN-γ, TNF and IL-2, and mobilisation of CD107 using intracellular cytokine assay as described previously (Smith C et al. Oncoimmunology 2017; 6(2): e1273311). Cells were acquired using a BD LSR Fortessa with FACSDiva software (BD Biosciences). Post-acquisition and Boolean analysis was performed using FlowJo software (FlowJo LLC, Ashland, Oreg.).

Results

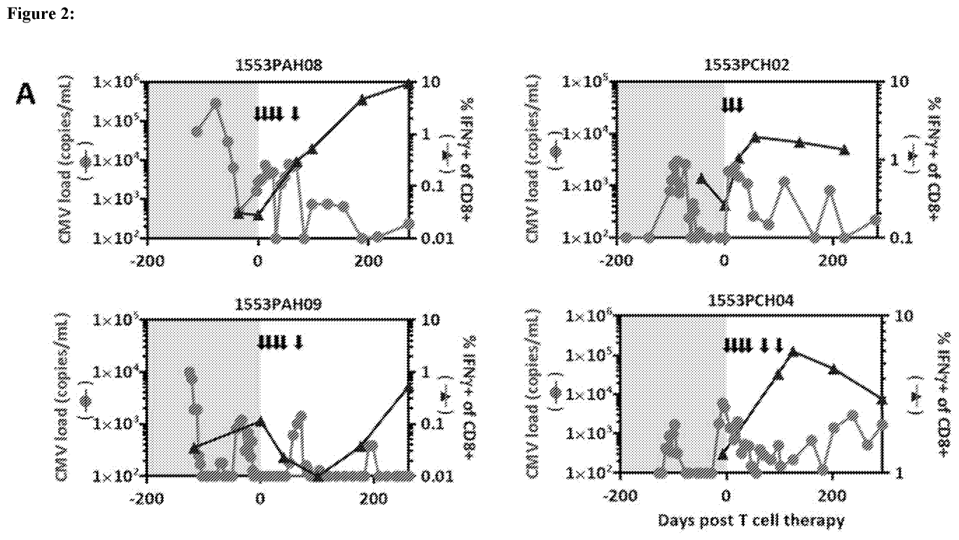

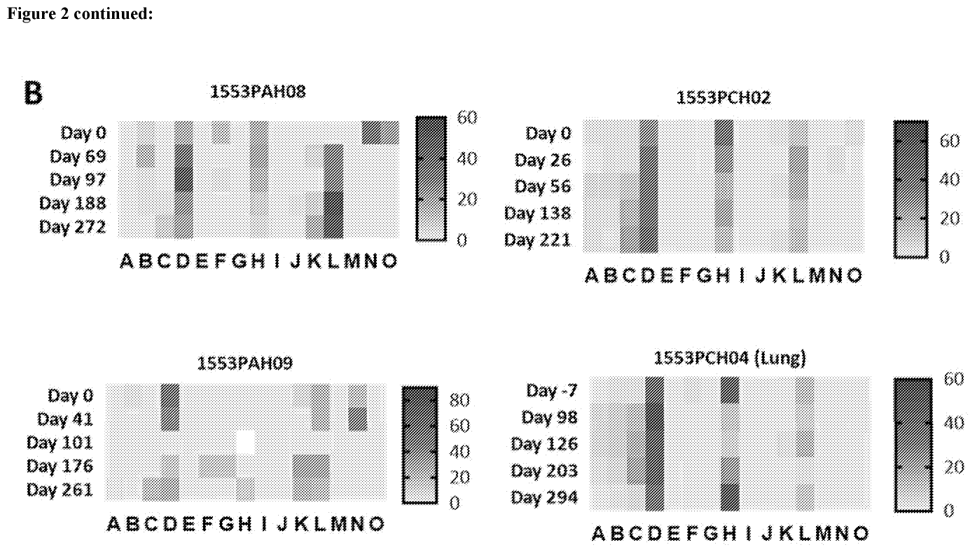

Representative data from four SOT patients who showed an objective response to adoptive immunotherapy are shown in FIG. 2. The shaded box represents the analysis period pre-treatment and the arrows represent each infusion of autologous in vitro-expanded CMV-specific T-cells. This analysis revealed evidence of immunological reconstitution post-therapy in association with control of viremia. This is best exemplified in patient 1553PAH08, whose proportion of IFN-γ-producing CMV-specific T-cells increased from 0.03% prior to the first infusion to 9.3% at the completion of the follow-up period, with a concordant reduction in viral load and cessation of anti-viral drug therapy (FIG. 2A). A similar improvement in peripheral T-cell immunity following the commencement of T-cell infusions was also evident in other patients including 1553PAH09, 1553PCH02 and 1553PCH04 (FIG. 2A). Immune reconstitution in these patients was observed in spite of the continuation of immunosuppressive therapies prescribed prior to adoptive T-cell therapy (Table 2). Coincident with immune reconstitution, improvement in the functional quality of CMV-specific T-cell responses was also observed, characterised by an increased proportion of T-cells co-expressing IFN-γ, TNF and CD107 (FIG. 2B). In contrast, patient 1553RAH01, who did not respond clinically to therapy, showed no evidence of immunological reconstitution post-therapy (data not shown). Follow-up immunological analysis was not possible in patient 1553PCH03, who died early after the commencement of therapy due to complications related to CMV infection. Although patients 1553PAH06 and 1553PCH05 showed clinical improvement, there was no change in the frequency of CMV-specific T-cells in their peripheral blood following adoptive T-cell therapy (data not shown).

### Example 5: Polychromatic Profiling of T-Cell Phenotype

To characterize the phenotypes of CMV-specific T-cell following adoptive T-cell therapy and reconstitution, T-cells acquired from each patient were incubated with allophycocyanin-labelled MHC class I multimers specific for the HLA-A2-restricted epitope NLV (pp65), the HLA-A1 restricted epitope VTE pp65), the HLA-B7 restricted epitopes TPR and RPH (pp65), or the HLA-B8 restricted epitopes ELR and ELK (IE-1). For assessment of surface phenotype, cells were then incubated for a further 30 minutes at 4° C. with the following antibodies anti-CD45RA FITC, anti-CD8 PerCP-Cy5.5, anti-CCR7 AF700, anti-CD95 BV421, anti-CD28 BV480, anti-CD57-Biotin followed by SA-BV605, anti-CD27 PE, anti-CD19 PE-Cy5, anti-CD4 PE-Cy7 and Live/Dead NIR; (Cells were acquired using a BD LSR Fortessa with FACSDiva software (BD Biosciences). Post-acquisition analysis was performed using FlowJo Software (TreeStar) and t-distributed stochastic neighbor embedding (tSNE) analysis to define immunological phenotypic changes post-therapy.

Results

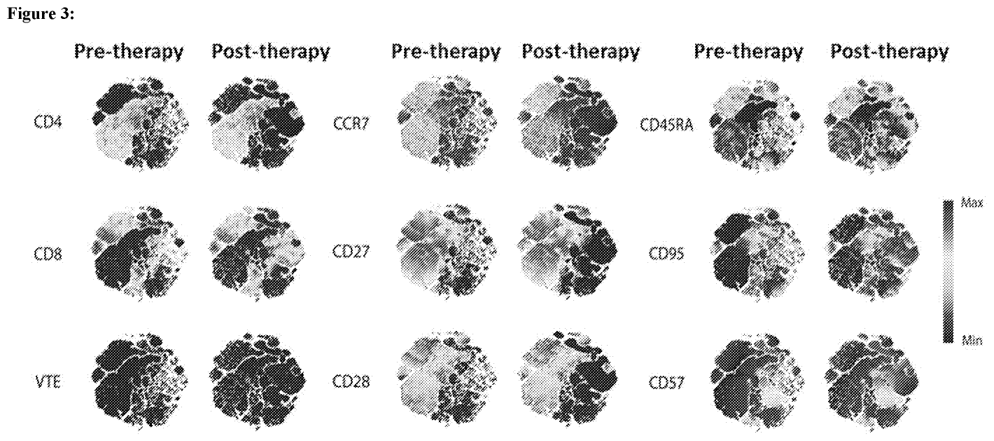

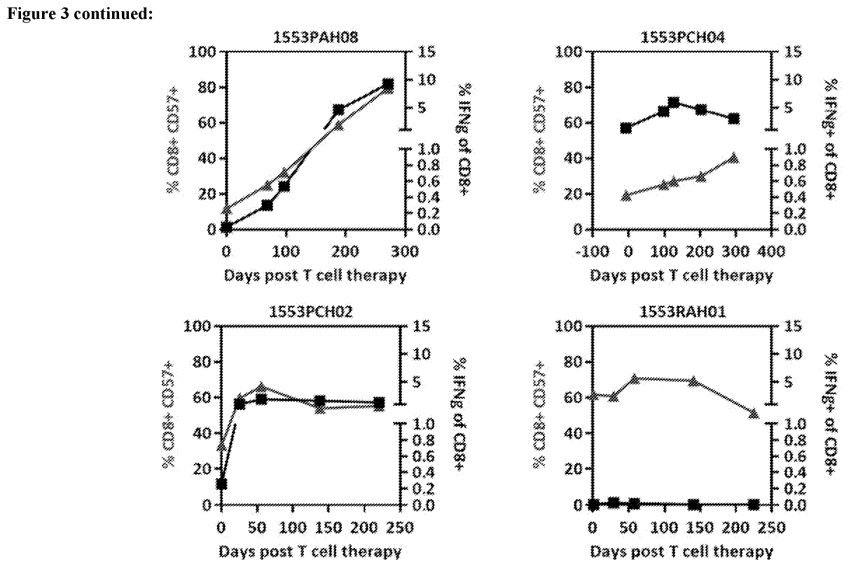

Representative tSNE analysis in the upper panels of FIG. 3 show the expression of T-cell phenotype markers and CMV-specific T-cells (VTE) pre-therapy and post-therapy in patient P1553PAH08 and demonstrate an increase in the expression of CD57. Data in the lower panels of FIG. 3 represent an overlay of the proportion of CD8+ T-cells expressing CD57 post T-cell therapy and the percentage CMV-specific IFN-γ producing cells in three SOT recipients (P1553PAH08, 1553PCH02 and 1553PCH04) who responded to adoptive T-cell therapy and one SOT recipient (P1553RAH01) who failed to show any clinical response.

### Concluding Summary

In contrast with CMV-specific T-cells generated from healthy CMV-seropositive individuals, for administration in hematopoietic stem cell transplantation (HSCT) recipients (Fuji et al. Current opinion in infectious diseases 2017; 30(4): 372-6; Tzannou et al. J Clin Oncol 2017; 35(31): 3547-57.), autologous CMV-specific immunotherapy in SOT recipients is dependent upon the capacity to generate CMV-specific T cells from immunosuppressed individuals. However, CMV-specific T-cells from 20 of the 21 patients, as disclosed herein, were successfully generated. Despite the heavy immunosuppressive regimes used to prevent graft rejection, the majority of the patients were able to prime a CMV-specific T-cell response and, in some cases, had a high precursor frequency in their PBMC prior to T-cell expansion. Functional defects were noted in the CMV-specific T cells in the peripheral blood of SOT recipients as recently reported (Snyder LD, Chan C, Kwon D, et al. Polyfunctional T-Cell Signatures to Predict Protection from Cytomegalovirus after Lung Transplantation. Am J Respir Crit Care Med 2016; 193(1): 78-85); characterized by a reduced capacity to express TNF and IFN-γ. Importantly, this phenotype could be reversed following in vitro stimulation, with the majority of expanded CMV-specific T cells co-expressing CD107a, TNF and IFN-γ.

Virological and immunological monitoring provided evidence of the potential benefit that immunological reconstitution following adoptive immunotherapy can have upon viral control in SOT patients. There was clear evidence in multiple patients that immune reconstitution coincided with reduction in, or resolution of, viral reactivation. This is of particular importance for the SOT recipients who had developed drug resistance, had ongoing CMV-associated end-organ disease, or a previous history thereof. Furthermore, the adoptive T-cell therapy disclosed herein could be safely used concurrently with immunosuppressive therapies for preventing CMV-associated complications in patients unable to tolerate standard anti-viral drug therapy.

1. A pool of immunogenic peptides comprising HLA class I and class II-restricted Cytomegalovirus (CMV) peptide epitopes capable of inducing proliferation of peptide-specific T cells, wherein the peptide pool comprises at least one of the epitope amino acid sequences set forth in SEQ ID NOs. 25 to 29, or combinations thereof.

2. A pool of immunogenic peptides comprising HLA class I and class II-restricted CMV peptide epitopes capable of inducing proliferation of peptide-specific T cells, and wherein the peptide pool comprises at least one peptide epitope derived from each of the CMV antigens pp50, pp65, IE-1, gB and gH.

3. (canceled)

4. The pool of immunogenic peptides of claim 1, comprising each of the CMV peptide epitope amino acid sequences set forth in Table 1.

5. (canceled)

6. (canceled)

7. A method of producing a preparation of polyfunctional, CMV-specific cytotoxic T cells (CTLs), comprising:
a) isolating a sample comprising CTLs;
b) exposing said sample to the pool of immunogenic peptides of claim 1; and
c) harvesting the CTLs.

8. (canceled)

9. The method of claim 7, wherein the sample comprising CTLs comprises peripheral blood mononuclear cells (PBMCs) from a healthy donor or an immunocompromised donor (e.g., undergoing immunosuppressive therapy, a solid organ transplant recipient, receiving anti-viral therapy).

10. (canceled)

11. (canceled)

12. (canceled)

13. (canceled)

14. The method of claim 7, wherein the exposed sample of step b) is incubated for at least 14 days.

15. The method of claim 7, further comprising incubating the exposed sample of step b) with IL-2 on day 0 or on day 2.

16. (canceled)

17. The method of claim 7, further comprising adding IL-2 every three days.

18. The method of claim 7, further comprising administering the CTLs to a subject suffering from a CMV infection.

19. (canceled)

20. CTLs prepared by the method of claim 7.

21. A method of treating or preventing CMV infection in a subject, comprising administering to the subject the CTLs of claim 20.

22. (canceled)

23. The method of claim 21, wherein the CTLs administered to the subject are autologous.

24. (canceled)

25. (canceled)

26. (canceled)

27. (canceled)

28. The method of claim 21, wherein at least 5%, at least 10%, at least 20%, at least 60%, or at least 90%, of the CTLs express CD107a.

29. (canceled)

30. (canceled)

31. (canceled)

32. (canceled)

33. The method of claim 21, wherein at least 5%, at least 10%, at least 20%, at least 60%, or at least 90%, of the CTLs express IFN-γ.

34. (canceled)

35. (canceled)

36. (canceled)

37. (canceled)

38. The method of claim 21, wherein at least 5%, at least 10%, at least 20%, at least 60%, or at least 90%, of the CTLs express TNF.

39. (canceled)

40. (canceled)

41. (canceled)

42. (canceled)

43. The method of claim 19, wherein at least 1%, at least 5%, at least 10%, or at least 20%, of the CTLs express IL-2.

44. (canceled)

45. (canceled)

46. (canceled)

47. The method of claim 21, wherein at least 20%, at least 43%, at least 55%, or at least 90%, of the CTLs express CD107a, IFN-γ, and TNF.

48.-92. (canceled)

93. A method of reducing CMV viral load in a subject that has received a solid organ transplant by administering to the subject the CTLs of claim 20.

94. A method of treating or preventing CMV-associated end organ disease in a subject that has received a solid organ transplant by administering to the subject the CTLs of claim 20.

95. A method of reducing or eliminating the need for anti-viral therapy in a subject that has received a solid organ transplant by administering to the subject the CTLs of claim 20.

96. (canceled)

97. (canceled)

98. (canceled)
